(19) United States
(12) Patent Application Publication
(10) Pub. No.: US 2019/0374681 A1
(43) Pub. Date: Dec. 12, 2019

(54) METHOD FOR ISOLATING HUMAN BRAIN TISSUE-DERIVED NEURAL STEM CELL AT HIGH EFFICIENCY

(71) Applicant: MEDINNO INC., Gyeonggido-do (KR)

(72) Inventors: Kyeung-Min JOO, Seoul (KR); Hyun NAM, Gyeonggi-do (KR); Do-Hyun NAM, Seoul (KR); Seung-Chyul HONG, Seoul (KR); Je-Young YEON, Seoul (KR)

(21) Appl. No.: 16/487,458

(22) PCT Filed: Feb. 23, 2018
(86) PCT No.: PCT/KR2018/002253

§ 371 Date: Aug. 21, 2019

(30) Foreign Application Priority Data
Feb. 24, 2017 (KR) 10-2017-0024657

(51) Int. Cl. A61L 27/38; C12N 5/0797

(52) U.S. Cl. CPC A61L 27/383; C12N 2509/00; C12N 5/0623

(57) ABSTRACT

The present invention relates to a method for cultivation and isolation of neural stem cells whereby neural stem cells can be rapidly proliferated on mass scale and isolated at high efficiency and to a stroke patient-derived human adult neural stem cell, cultured and isolated thereby, for use in transplantation.

### TECHNICAL FIELD

The present invention relates to a method of culturing neural stem cells and isolating the neural stem cells at high efficiency, and human adult neural stem cells for transplantation which are cultured and isolated thereby.

### BACKGROUND ART

Stroke is defined as a disorder of blood flow to brain tissue. This may be a disorder at a particular site of the brain or, for example, may also be a substantial decrease in all flow when pumping of the heart is completely impaired. More frequently, a stroke occurs when a flow to a specific site of the brain is interrupted by clogging or rupture of blood vessels passing through the brain. Blockage of blood vessels generally occurs in the arteries supplying blood to the brain, for example, through the formation of an embolism or thrombus, which is known as an ischemic stroke.

Stroke is the third leading cause of death in developed countries. In the United States, treatment costs and productivity loss incurred as a result of stroke death are estimated at approximately 40 trillion dollars. Meanwhile, the only method of effectively treating stroke is to administer a thrombolytic agent such as a tissue plasminogen activator (t-PA), which enzymatically cleaves a thrombus-producing protein to eliminate the thrombus. However, administration of t-PA may cause side effects such as bleeding when it is too late, and thus t-PA may be administered only within 3 hours after the first symptom appears. Moreover, t-PA can only be used for an ischemic stroke, and when administered for a hemorrhagic stroke, side effects such as death may generally occur. These stroke treatment methods are merely for emergency treatment or delaying the progression of the disease, but there is still no fundamental treatment measure.

Stem cells, which can differentiate into various types of tissues in the body, i.e., undifferentiated cells, are capable of differentiating into various tissue cells under appropriate conditions, and thus may be applied to treatment such as regeneration of damaged tissues and the like. A stem cell therapeutic agent is expected to enable the regeneration of injured nerves and is known to be involved in regeneration by secreting various substances that improve damaged environments, as well as direct regeneration. Currently, research on mesenchymal stem cells is the most clinically advanced, but there are conflicting opinions on the effectiveness thereof for the treatment of stroke or other neurodegenerative diseases. Although clinical trials of fetus-derived neural stem cells for patients with stroke and spinal injuries are ongoing, there are many technical problems as well as ethical issues.

### DISCLOSURE

### Technical Problem

Therefore, the inventors of the present invention discovered that neural stem cells isolated from brain tissue extracted during surgery of an emergency stroke patient could be rapidly mass-proliferated and isolated with high efficiency by being cultured under specific conditions, and thus completed the present invention.

### Technical Solution

To achieve the above object, the present invention provides a method of culturing a neural stem cell, including: collecting cells from brain tissue; treating the collected cells with collagenase and DNase I, or papain, cysteine, and DNase I to isolate single cells; dividing the single cells into two or more tubes, mixing the single cells in each tube with Percoll, and centrifuging each mixture to recover cells; primary culturing the recovered cells; and sub-culturing the primary cultured cells.

In one embodiment of the present invention, the brain tissue may be brain tissue extracted during surgery on an emergency stroke patient, for example, the tissue extracted from the brain or spinal cord. For example, the brain tissue may include a tissue from temporal lobe epilepsy, a stroke surgical sample (including temporal lobe), trauma tissue (including temporal lobe), and the like.

In one embodiment of the present invention, the neural stem cell may be a human adult neural stem cell for transplantation which is derived from a stroke patient. The human adult neural stem cell may be autotransplanted or allotransplanted.

In one embodiment of the present invention, the primary culturing and/or sub-culturing may be performed using an adherent culture method.

In one embodiment of the present invention, the sub-culturing may be performed three times or less.

The present invention also provides a human adult neural stem cell for transplantation derived from brain tissue which is cultured by the method of culturing a neural stem cell according to the present invention.

### Advantageous Effects

The present invention can not only address ethical issues by isolating and culturing a neural stem cell from brain tissue of a stroke patient himself or herself, but also can readily provide a transplantable human adult neural stem cell.

According to the present invention, neural stem cells can be rapidly mass-proliferated under specified isolation and culture conditions. In particular, single cells isolated from brain tissue can be divided into two tubes and mixed with Percoll to recover cells, thereby increasing the yield of neural stem cells by about 2 times. In addition, the primary culture or sub-culture can be performed using an adherent culture method, thus stably increasing culture efficiency and increasing purity, as compared to an existing sphere method. In addition, a sufficient number of cells to be transplanted into a patient can be cultured through performance of sub-culturing three times or less, thus enabling rapid proliferative culture, particularly, rapid transplantation of the cells into an emergency stroke patient.

### DESCRIPTION OF DRAWINGS

FIG. 1 illustrates a process of culturing a neural stem cell according to an embodiment of the present invention.

FIG. 2 illustrates the number of cells according to Percoll treatment, according to an embodiment of the present invention.

FIG. 3 illustrates a method of differentiating a neural stem cell according to an embodiment of the present invention.

FIG. 4 is a set of images showing results of confirming the ability of a neural stem cell to differentiate into a neuron and an astrocyte according to differentiation conditions, according to an embodiment of the present invention.

### MODES OF THE INVENTION

The present invention provides a method of culturing a neural stem cell, including: collecting cells from brain tissue; treating the cells with collagenase and DNase I, or papain, cysteine, and DNase I to isolate single cells; dividing the single cells into two or more tubes, mixing the single cells in each tube with Percoll, and centrifuging each mixture to recover cells; primary culturing the recovered cells; and sub-culturing the primary cultured cells.

The neural stem cell of the present invention is isolated from brain tissue extracted during surgery on a stroke patient, and may be autotransplanted into the stroke patient or allotransplanted.

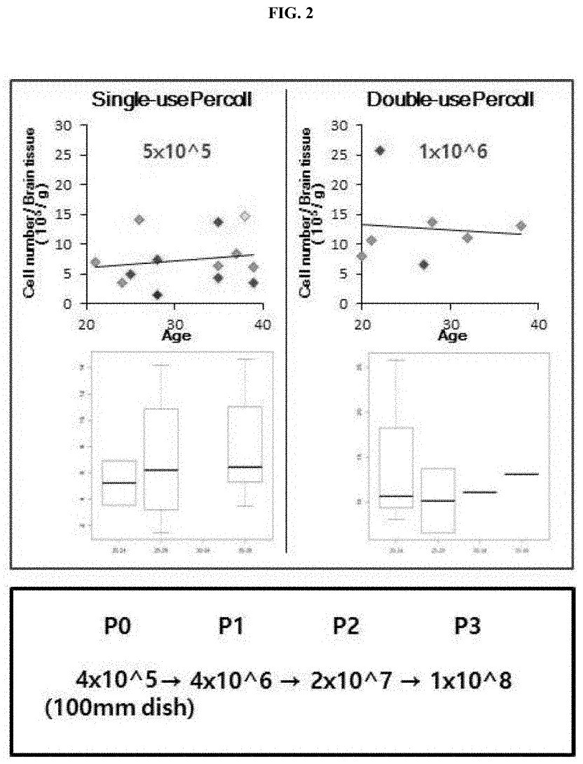

Generally, single cells isolated from a biological tissue are subjected to a process of removing impurities using Percoll. In the method of the present invention, the single cells may be divided into two tubes and mixed with Percoll, followed by centrifugation to recover cells, whereby cell yield is increased about 2-fold compared to a case in which single cells are mixed with Percoll in a conventional single tube (see FIG. 2).

A medium used for primary culturing or sub-culturing of the present invention may be any medium generally used for culturing stem cells. For example, a medium containing serum (e.g., fetal bovine serum, horse serum, and human serum) may be used. The medium that may be used in the present invention may be, for example, the RPMI series such as RPMI 1640 (Moore et al., J. Amer. Med. Assoc. 199:519(1967)), Eagles's MEM (Eagle's minimum essential medium, Eagle, H. Science 130:432(1959)), α-MEM (Stanner, C. P. et al., Nat. New Biol. 230:52(1971)), Iscove's MEM (Iscove, N. et al., J. Exp. Med. 147:923(1978)), 199 medium (Morgan et al., Proc. Soc. Exp. Bio. Med., 73:1(1950)), CMRL 1066, F12 (Ham, Proc. Natl. Acad. Sci. USA 53: 288 (1965)), F10 (Ham, R.G. Exp. Cell Res. 29: 515 (1963)), DMEM (Dulbecco's modification of Eagle's medium, Dulbecco, R. et al., Virology 8: 396 (1959)), a mixture of DMEM and F12 (Barnes, D. et al., Anal. Biochem. 102: 255 (1980)), Way mouth's MB752/1 (Waymouth, C. J. Natl. Cancer Inst. 22: 1003 (1959)), McCoy's 5A (McCoy, T. A., et al., Proc. Soc. Exp. Biol. Med. 100:115 (1959)) and the MCDB series (Ham, R.G. et al., In Vitro 14:11 (1978)), but the present invention is not limited thereto. The medium may include other components, for example, an antibiotic or antifungal agent (e.g., penicillin and streptomycin), glutamine, and the like.

Sub-culturing of stem cells is generally performed seven or nine times or more. In the method of the present invention, however, due to mixing with Percoll in two tubes and the use of an adherent culture method, the sub-culturing process may be performed three times or less, whereby a sufficient amount of neural stem cells for transplantation may be acquired.

Advantages and features of the present invention, and methods of achieving them will become apparent with reference to embodiments described below in detail. Hereinafter, the present invention will be described in further detail with reference to the following examples. However, these examples are provided to specifically explain the present invention and are not intended to limit the scope of the present invention.

### EXAMPLE 1. ACQUISITION OF NEURAL STEM CELLS

### Isolation and Culture of Human Neural Stem Cells

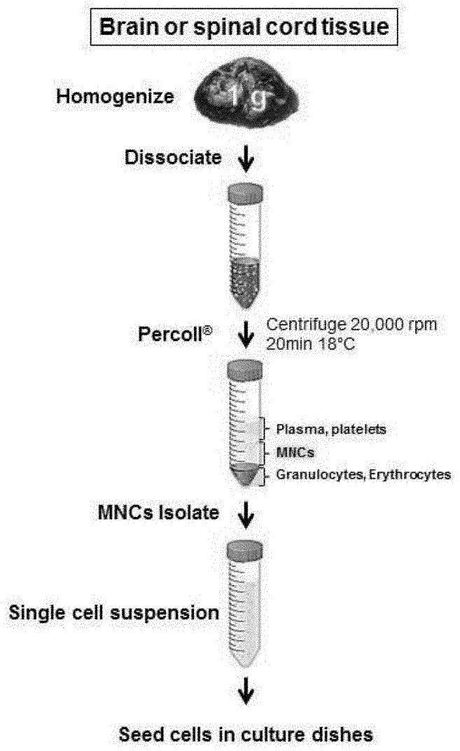

Brain tissue (brain tissue in an outer ceiling area of a lateral ventricle (brain tissue in the pathway for bleeding removal or ventricular puncture)) removed through a surgical operation (Department of Neurosurgery, Samsung Medical Center) from a stroke patient was obtained, and cultured according to processes as illustrated in FIG. 1.

First, the obtained brain tissue was immersed in a solution prepared by adding 3% antibiotic-antimycotic (Gibco) or 3% penicillin/streptomycin (Gibco) to Hank's balanced salt solution (HBSS, Welgene) and stored, and within a maximum of 12 hours after surgery, cells were isolated. In the case of difficulty in immediately performing cell isolation, the resulting brain tissue was kept refrigerated at 4□ before being subjected to cell isolation.

The obtained brain tissue was weighed and then rinsed two or three times with a sterilized PBS solution, and then mechanically pulverized with scissors or a razor blade, and stored in an enzyme solution prepared by mixing Collagenase (0.4 mg/ml, Gibco) and DNase I (0.01 mg/ml to 1 mg/ml, Roche) or mixing papain (10 unit/ml, Sigma), DL-Cysteine (400 ng/ml, Sigma), and DNase I (0.01 mg/ml to 1 mg/ml, Roche) in a CO₂ incubator at 37□ for 1 hour. Thereafter, the enzyme solution was treated with DMEM:F12(Gibco) and a 1% FBS solution in an amount equal to or greater than the enzyme solution to inactivate the enzymes, followed by pipetting with a pipette, pulverization, and passing through a 70 μM nylon mesh to obtain single cells.

Percoll (Sigma) was warmed in a 37□ water bath for about 5 minutes, and then 9 mL of Percoll and 1 mL of 10× PBS were added to a 50 mL sterile ultracentrifuge tube to adjust a concentration thereof to 1×. The obtained single cell suspension was diluted with 1× PBS to a total volume of 40 mL, and then divided into two 50 mL conical tubes at a volume of 20 mL each, and Percoll was added to each tube to adjust a total volume of each tube to 30 mL. Then, each tube was centrifuged at 20,000 rpm and 18□ for 20 minutes to remove erythrocytes and other tissues and cells. A white layer formed after centrifugation was separated using a pipette, and then washed twice with a DMEM:F12(Gibco) solution.

The final cells were suspended in a DMEM:F12 (Gibco) solution-based culture solution containing 0.5% to 1% FBS, a 1× B27 supplement (Gibco), bFGF (R&D), and EGF (R&D), the number of the cells was confirmed, and then 100 pi dishes were pre-treated with poly-L-ornithine (Sigma) before cell culture, followed by culturing to a density of 4×10⁵ cells/dish. At this time, only half of the culture solution was replaced at intervals of 3 days to 4 days, and 10 days to 14 days were generally taken until primary sub-culturing.

As a comparative example, neural stem cells were cultured in the same manner as in the above example, except that single cells were mixed with Percoll in a single tube, and the result was compared with that of the above example in which Percoll was mixed in two tubes. As can be seen in FIG. 2, while the number of cells was 5×10⁵ in the case in which Percoll was mixed in a single tube, 1×10⁶ cells were shown in the case in which Percoll was mixed in two tubes according to the present example.

### Subculture

The cells were sub-cultured when the cells occupied about 70% to 80% of a total area of each dish in the above culture process.

First, the existing cell culture solution was removed, followed by washing once with DPBS. Then, the cells were treated with 0.05% Trypsin/EDTA (T/E, Gibco) or Accutase such that the cells were immersed therein, stored in a 5% CO₂ incubator at 37□ for 2 minutes to 3 minutes, and then treated with a solution to which DMEM:F12 (Gibco) and 1% FBS were added, to inactivate the enzyme. The cells were pelleted using a centrifuge, and then the supernatant was removed and suspended in the cell culture solution.

The cells were counted and then 4×10⁵ cells were placed in each of 100 pi dishes and cultured. When sub-cultured once, the number of cells was increased by average of 10 times and 10³-fold cells were obtained when sub-culturing was performed three times.

The average time for sub-culturing once is 3 days to 4 days and sub-culturing three times may be done within 2 weeks, and thus 1×10⁸ cells were obtained within one month.

When sub-culturing is performed 7 times or more, the growth of cells slows down and there are many cases in which cells take a form similar to aging such as cells increasing in length and the like, and accordingly, the characteristics of stem cells are gradually lost. In addition, long-term culture causes an increase in genetic mutations.

When culture is performed using the method of the present invention, a sufficient number of cells for multi-dose administration and autotransplantation may be obtained performing sub-culturing three times or less.

### EXAMPLE 2. DIFFERENTIATION OF NEURAL STEM CELLS

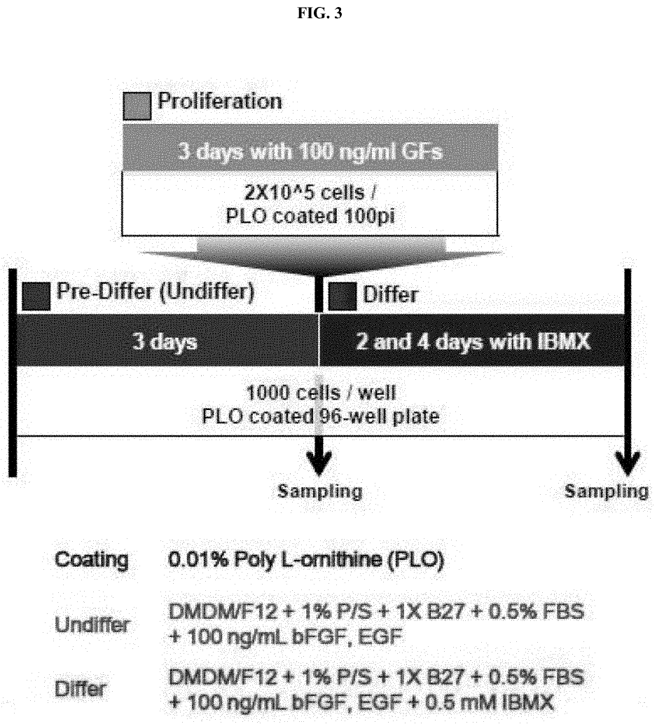

To confirm a differentiation ability of the neural stem cells obtained in Example 1, as illustrated in FIG. 3, the neural stem cells were differentiated.

First, the neural stem cells were cultured on poly-L-ornithine (PLO)-coated culture dishes for 3 days, and when the cells occupied 70% to 80% of a total area of each dish, the culture medium was replaced with a differentiation medium (DMEM/F12, 1% P/S, 1×B27, 0.5% FBS, 100 ng/mL bFGF, 100 ng/mL EGF, and 0.5 mM IBMX). On day 2 and day 4 after differentiation, the cells were immobilized and immunofluorescence-stained with Nestin, which is an undifferentiation marker, MAP2, which is a neuron-specific marker, and GFAP, which is an astrocytic marker, and then observed with a fluorescence microscope.

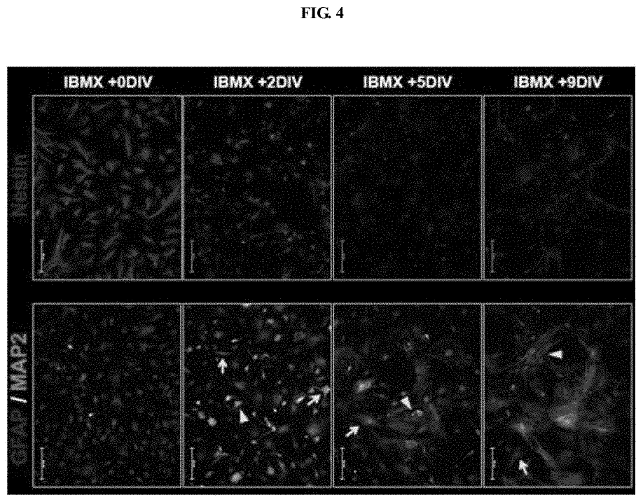

As can be seen in FIG. 4, it was confirmed that the neural stem cells differentiated into neurons and astrocytes. The ability of the neural stem cells to differentiate into neurons (MAP2+) and astrocytes (GFAP+) was confirmed under differentiation conditions, i.e., before differentiation (0 DIV) and after differentiation (2, 5, 9 DIV), and it was also confirmed that the expression of Nestin, which is an undifferentiation marker, was reduced as differentiation proceeded.

The present invention has been described with reference to exemplary embodiments thereof. It will be understood by those skilled in the art that various changes may be made in forms and details without departing from the essential features of the present invention. Therefore, the disclosed embodiments should be considered in an illustrative sense rather than a restrictive sense. The scope of the present invention is defined by the appended claims rather than by the foregoing description, and all differences within the scope of equivalents thereto should be construed as being within the scope of the present invention.

1. A method of culturing a neural stem cell, the method comprising:
collecting cells from brain tissue;
treating the collected cells with collagenase and DNase I, or papain, cysteine, and DNase Ito isolate single cells;
dividing the single cells into two or more tubes, mixing the single cells in each tube with Percoll, and centrifuging each mixture to recover cells;
primary culturing the recovered cells; and
sub-culturing the primary cultured cells.

2. The method of claim 1, wherein the neural stem cell is a human adult neural stem cell for transplantation which is derived from a stroke patient.

3. The method of claim 1, wherein the primary culturing or the sub-culturing is performed using an adherent culture method.

4. The method of claim 1, wherein the sub-culturing is performed three times or less.

5. A human adult neural stem cell for transplantation which is derived from brain tissue cultured using the method according to any one of claims 1.
